(19) United States
(12) Patent Application Publication
(10) Pub. No.: US 2012/0208172 A1
(43) Pub. Date: Aug. 16, 2012

(54) Methods of Increasing Liver Proliferation

(75) Inventors: Seth J. Karp, Chestnut Hill, MA (US); Martin Dib, Brookline, MA (US); Nhue Do, Jamaica Plain, MA (US)

(73) Assignee: Beth Israel Deaconess Medical Center, Inc., Boston, MA (US)

(21) Appl. No.: 13/345,823

(22) Filed: Jan. 9, 2012

Related U.S. Application Data
(63) Continuation-in-part of application No. PCT/US2010/041480, filed on Jul. 9, 2010.
(60) Provisional application No. 61/225,388, filed on Jul. 14, 2009.

(51) Int. Cl. A01N 1/02; C12N 5/02; C12N 5/071; C07D 487/04

(52) U.S. Cl. 435/1.1; 544/281; 435/375; 435/325

(57) ABSTRACT

The present invention is directed to methods of enhancing liver repair after injury, resection or transplantation using antagonists of the bone morphogenetic protein (BMP) signaling pathway in the liver.

### RELATED APPLICATIONS

This application is a continuation-in-part of International Application No. PCT/US2010/041480, which designated the United States and was filed on Jul. 9, 2010, published in English, which claims the benefit of U.S. Provisional Application No. 61/225,388, filed on Jul. 14, 2009. The entire teachings of the above applications are incorporated herein by reference.

### GOVERNMENT SUPPORT

The invention was supported, in whole or in part, by a grant K08 from the National Institute of Diabetes and Digestive and Kidney Diseases. The Government has certain rights in the invention.

### BACKGROUND OF THE INVENTION

The field of regenerative medicine has strived to improve the survival rates of patients with liver failure or insufficient liver mass after liver injury or resection surgery. In few clinical scenarios is the line between success and failure as stark as when treating patients with these conditions: successful liver recovery after injury usually brings with it complete restoration of health, whereas failed recovery results in the need for liver transplantation or death. Even a minute increment of improvement in liver regeneration or repair may be sufficient to change the outcome from death to full restoration of health.

In patients with acute liver failure, the ability to enhance repair could convert a non-recoverable liver condition into one in which the affected liver could fully return to health. This group includes twenty thousand (20,000) patients per year in the United States with Tylenol® (acetaminophen) overdose, acute viral hepatitis or other infectious causes, metabolic disorders, or toxic insults (Hoofnagle et al., 1995). Patients suffering from these illnesses are often young and with few other medical conditions, making a full recovery highly likely if the affected liver could be induced to recover or regenerate.

A group of patients undergoing liver surgery may experience small-for-size syndrome. This occurs after resection for cancer or after transplantation when using whole organs, split organs, or live-donors. In many of these cases, the residual liver mass is inadequate to support life. This small-for-size syndrome manifests as graft dysfunction and may lead to death (Tucker and Heaton, 2005).

Patients with liver cancer are generally not candidates for liver resection due to the difficulty in predicting the resultant liver mass after resection. Of the more than five hundred thousand (500,000) patients worldwide diagnosed with liver cancer each year (Parkin et al., 2002), nearly 80% of them are determined to be unresectable at the time of diagnosis (Zhu et al., 2006). If resection is performed, however, these patients will likely develop hyperbilirubinemia (i.e., jaundice) and ultimately liver failure. Since other options for cancer patients are severely limited, potential therapies to enhance or increase liver regeneration or repair would greatly increase the possibility of liver resection for many more patients who are with otherwise unresectable liver cancer. Development of pharmacological therapies which would make more of these patients resectable will have a profound impact in treatment of liver cancer.

One critical consideration during living donor liver transplantation is the size of the remaining donor liver as well as the size of the graft being transplanted to the recipient. In adult-to-child liver donation, the donor operation is a left lateral segmentectomy which is known to be a safe operation. In contrast, during adult-to-adult living donor liver transplantation, necessary use of a full right or left lobectomy poses a great risk to the donor for the remaining liver may not be sufficient to support life (Pomposelli et al., 2006). Enhancing liver regeneration, therefore, would revolutionize adult-to-adult living donor liver transplantation by allowing the use of smaller size of grafts.

Despite the great achievement of liver transplantation as a therapy for liver failure and scientific knowledge of molecular pathways involved in liver homeostasis, a pharmacological treatment and method for enhancing liver repair and regeneration is still in great need of development.

### SUMMARY OF THE INVENTION

The present invention elucidates biochemical events underpinning the process of liver repair and regeneration and provides methods for enhancing hepatocyte proliferation, increasing liver regeneration and preventing and treating liver injury for the broad variety of communities of patients in various clinical settings.

The present invention is based on a discovery that the bone morphogenetic protein (BMP) signaling pathway is an active inhibitor of hepatocyte proliferation and that treatment with an antagonist of the pathway results in enhancement of hepatocyte proliferation and increased liver regeneration after liver injury in vivo.

In a first aspect, the present invention provides methods of increasing proliferation of hepatocytes by contacting the hepatocytes with an effective amount of an antagonist of the bone morphogenetic protein (BMP) signaling pathway. In one embodiment, the antagonist of the BMP signaling pathway inhibits or down regulates a constitutively active BMP signaling pathway. For example, the BMP signal pathway is a BMP2 or BMP4-mediated signaling pathway. In yet another embodiment, the antagonist of the BMP signaling pathway inhibits type I BMP receptor signal transduction. In one embodiment, the antagonist of the BMP signaling pathway inhibits the type I BMP receptor signal transduction by binding to the type I receptor. In another embodiment, the type I receptor includes ALK2, ALK3 or ALK6. In another embodiment, the antagonist of the BMP signaling pathway inhibits phosphorylation of SMAD 1, 5 or 8. In one embodiment, the antagonist of the BMP signaling pathway inhibits binding of BMP2 or 4 to a BMP receptor or interaction of a type II BMP receptor with a type I BMP receptor. In another embodiment, the antagonist of the BMP signaling pathway is a chemical agent. The chemical agent can be dorsomorphin, LDN193189 or an analogue thereof.

In a second aspect, the present invention is directed to methods of increasing liver regeneration after partial loss or injury to the liver in a mammalian subject in need thereof by administering to the subject an effective amount of an antagonist of the bone morphogenetic protein (BMP) signaling pathway. In one embodiment, the partial loss or injury to the liver is caused by a surgical or transplantation procedure such as liver resection. In another embodiment, the subject in need suffers from liver insufficiency after resective liver surgery. In one embodiment, the subject in need is a mammal such as a human. For example, the subject in need is a liver transplantation recipient or transplantation donor.

In yet another embodiment, the antagonist of the BMP signaling pathway inhibits or down regulates a constitutively active BMP signaling pathway. In one embodiment, the BMP signal pathway is a BMP2 or BMP4-mediated signaling pathway. In one embodiment, the antagonist of the BMP signaling pathway inhibits type I BMP receptor signal transduction. In one embodiment, the antagonist of the BMP signaling pathway inhibits the type I BMP receptor signal transduction by binding to the type I BMP receptor. In one embodiment, the type I BMP receptor includes ALK2, ALK3 or ALK6. In one embodiment, the antagonist of the BMP signaling pathway inhibits phosphorylation of SMAD 1, 5 or 8. In yet another embodiment, the antagonist of the BMP signaling pathway inhibits binding of BMP2 or 4 to a BMP receptor or interaction of a type II BMP receptor with a type I BMP receptor. The antagonist of the BMP signaling pathway is a chemical agent. Chemical agents may include, but are not limited to, dorsomorphin, LDN193189 and an analogue thereof.

In another aspect, the present invention includes methods of preventing or treating a liver injury in subjects that would benefit by administering effective amount of an antagonist of the bone morphogenetic protein (BMP) signaling pathway. The antagonist of the BMP signaling pathway inhibits or down regulates a constitutively active BMP signaling pathway as described above. The liver injury can be caused by a hepatotoxic chemical agent. The hepatotoxic chemical agent may include acetaminophen. In one embodiment, the liver injury is caused by a liver disease including, for example, hepatitis viral infections, autoimmune hepatitis, cirrhosis, acute or chronic liver failure, or cancer.

In yet another aspect, the present invention provides methods of enhancing liver regeneration in a subject in need thereof by administering to the subject an effective amount of an antagonist of the bone morphogenetic protein (BMP) pathway in which the antagonist inhibits BMP2 or BMP4-mediated phosphorylation of SMAD 1, 5 or 8 in the liver. In one embodiment, the antagonist is administered to the subject prior to partial loss or injury to the liver.

In yet another aspect, the present invention relates to a method of increasing proliferation of hepatocytes by contacting the hepatocytes with an effective amount of dorsomorphin or LDN193189 which inhibits BMP2 or BMP4-mediated phosphorylation of SMAD 1, 5 or 8 in hepatocytes.

Determining the fundamental elements of how the liver responds to injury has been difficult in the past. Although there are numerous molecular pathways implicated in liver homeostasis, there has been no effective therapy for patients with liver injury or inadequate liver mass after resection, particularly by enhancing liver regeneration and repair. A signal transduction pathway as described herein, whose function is to negatively control liver growth by constitutively inhibiting hepatocyte proliferation would help solve a fundamental riddle of how the liver senses its size and regulates its growth and can provide a unique opportunity for a potential therapy. More importantly, such a signaling pathway can serve as a useful target for potential agents, leading to successful development of an effective therapy for patients with liver injury or inadequate liver mass after resection.

### BRIEF DESCRIPTION OF THE DRAWINGS

FIG. 1 depicts expression levels of bone morphogenetic protein 4 (BMP4) in mouse as quantified by real time polymerase chain reaction (RT-PCR) after two-thirds (“⅔”) hepatectomy. Relative expression levels of BMP4 as compared to control (i.e., glyceraldehyde 3-phosphate dehydrogenase (GAPDH)) are shown (p<0.05 for 12-24 hour time points).

FIG. 2 depicts relative amount of RT-PCR products of BMP4 at 0 hour and 36 hour time points in mice as compared to control, GAPDH.

FIG. 3 depicts expression pattern of ALK3 protein, a type I BMP receptor for BMP2 and BMP4 ligands, in the liver of a mouse (PV: portal vein, HN: hepatocyte nucleus) as shown by immunohistochemistry using a monoclonal antibody directed against ALK3 protein.

FIG. 4 depicts Western blot analysis of BMP4 protein expression in the liver of floxed BMP4 transgenic mice exposed to Cre-recombinase (i.e., “BMP4 null”) as compared to BMP4 protein expression in the wild-type liver.

FIG. 5 depicts Ki67 proliferation index after ⅔ hepatectomy for BMP4 null mice and wild-type littermates (p<0.05).

FIG. 6 depicts BrdU proliferation index after ⅔ hepatectomy for 8 week old mice treated with dorsomorphin (10 mg/kg) or saline as control.

FIG. 7 depicts the remnant liver as a percentage of the total body weight as measured at 36 and 48 hours following hepatectomy.

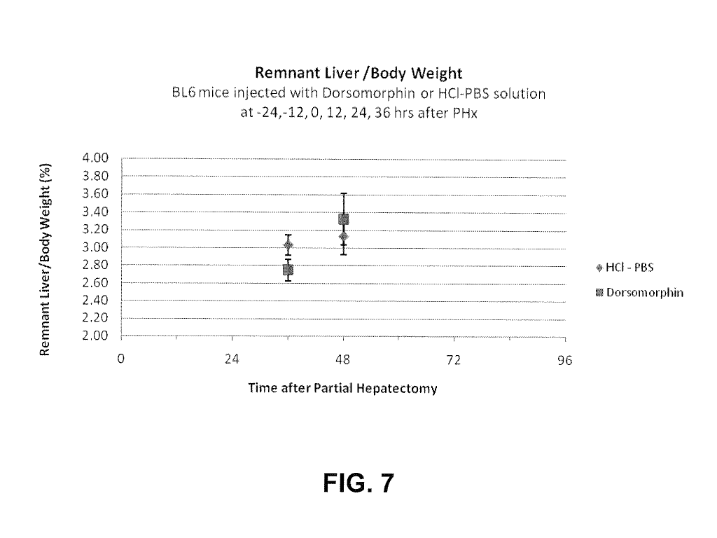

FIG. 8 depict a schematic illustration of analogues of dorsomorphin.

### DETAILED DESCRIPTION OF THE INVENTION

It has now been discovered that the bone morphogenetic protein (BMP) signaling pathway is constitutively active in the mammalian liver and that, when the BMP signaling pathway active, the pathway functions to repress or inhibit hepatocyte proliferation in the liver. It has been found that administration of an antagonist of the BMP signaling pathway, for example, a drug inhibitor of a BMP signaling, can enhance hepatocyte proliferation and increase liver repair and regeneration after liver injury.

Bone morphogenetic proteins (BMPs) are a family of proteins, which were originally identified and characterized by their ability induce cartilage and bone formation in vivo (Reddi, A. H. (1992) Curr, Opin. Cell Biol. 4, 850-855). Over twenty BMPs have been identified to date and reported to be recognized by two different serine-threonine kinase receptors: the type I and type II BMP receptors. At least three different type I serine-threonine kinase receptors and at least three different type II serine-threonine kinase receptors have been identified. The three identified type I BMP receptors are activin receptor-like kinase-2 (ALK2) (also known as activin receptor-I), ALK3 (also known as BMPR-IA), and ALK6 (also known as BMPR-IB) and the other three identified type II BMP receptors include BMPRII, ActRIIa and ActRIIb.

Both type I and type II BMP receptors are capable of transducing extracelluar BMP signals into the cytoplasm of the target cells which express the receptors on their plasma membrane. Dimeric BMP ligands are known to facilitate assembly of the type I and II receptors to form a heterodimer and this heterodimerization of the receptors allows type II receptor to phosphorylate the type I receptor. When phosphorylated, the type I BMP receptor is shown to phosphorylate BMP-responsive SMAD effectors, for example, SMAD1, SMAD5 and SMAD8. Phosphorylation of these effectors in turn results in nuclear translocation of the effectors in complex with SMAD4. In the nucleus of the target cell, phosphorylated SMADs upregulate expression of a set of genes responsive to the BMP signals, including, but not limited to, Id-1. The BMPs are also capable of eliciting SMAD-independent signaling via the type II BMP receptor, which, upon binding with a BMP, can independently activate the mitogen-activated protein kinase (MAPK) p38 mediated response.

All BMPs contain the characteristic seven highly conserved cysteines in their carboxyl-terminal portions and, thus, belong to the transforming growth factor-β (TGF-β) superfamily such as TGF-βs, activins, inhibins, and Müllerian inhibiting substances (Massague, J. (1990) Annu. Rev. Cell Biol. 6, 597-641). Among the BMPs, BMP2, BMP3, BMP4, BMP5, BMP6 and BMP7 have been the focus of studies. There are several representative members of these BMPs. BMP2 and BMP4 are closely related to one another by having approximately 90% amino acid identity. However, their sequences differ from those of BMP3, BMP5, BMP6 and BMP7. The second group, BMP5, BMP6 and BMP7, exhibit approximately 80% amino acid identity to each other, leaving BMP3 and others as the third group of this protein family.

The BMPs are known to bind both type I and type II BMP receptors. For example, BMP2 exhibits a high specificity for both type I and type II BMP receptors (Keller, S. et al. (2004) Nature Structural and Molecular Biology, 11:481-488). Notwithstanding, type II receptor utilization differs significantly between BMP2 or 4 and BMP6 or 7. A greater reliance on BMPRII is observed for BMP2 or 4 relative to BMP6 or 7, whereas ActRIIa is more critical to signaling by BMP6 or 7 than BMP2 or 4 (Layery, K. et al., (2008) J. Biol. Chem. 283:20948-20958). Significant differences were also observed for the type I receptors. ALK3 and ALK6 are more frequently utilized by BMP2 and BMP4, whereas ALK2 is the preferred type I receptor for BMP6 or BMP7 (Layery, K. et al., (2008) J. Biol. Chem. 283:20948-20958; Ro et al., (2004) Oncogen, 23:3024-3032).

The present invention relates to methods of enhancing proliferation of hepatocytes by inhibiting BMP signaling using an antagonists of the BMP signaling pathway. The present invention relates to methods of enhancing mammalian liver regeneration by inhibiting BMP signaling using an antagonists of the BMP signaling pathway. The present invention includes methods of enhancing liver repair by inhibiting BMP signaling using an antagonists of the BMP signaling pathway. The present invention relates to methods of preventing and treating liver injury in a subject in need thereof by administering an effective amount of an antagonist of the bone morphogenetic protein (BMP) signaling pathway in the liver.

As used herein, a “liver injury” includes, but is not limited to, hepatectomy, resection of the liver, liver transplantation, or other physical trauma or conditions caused by surgical removal of a portion of the liver. Liver injury relating to the acute liver insufficiency can be caused by resection of hepatocellular carcinoma, liver transplantation, or other acute physical traumas or conditions caused by surgical removal of a portion of the liver. A “liver injury” as used herein also includes, but is not limited to, hepatic tissue injury or damage, or loss of hepatic cells or tissue by apoptosis, necrosis or other physical conditions caused by a hepatotoxic agent or compound. Liver injury relating to the acute liver insufficiency can be also due to overdose of one or more pharmaceutical agents (e.g., acetaminophen/paracetamol) or ingestion of toxic amount of one or more hepatotoxic chemical agents. Alternatively, a “liver injury” as used herein also includes, but is not limited to, tissue injury or damage, or loss of hepatic cells or tissue by apoptosis, necrosis or other physical conditions caused by a liver disease including, but not limited to, hepatitis A, hepatitis B, hepatitis C, other hepatitis viral infections, autoimmune hepatitis, cirrhosis, biliary cirrhosis, acute liver failure, acute hepatotoxicity, chronic liver failure, acute liver infection, cancer of the liver, Wilson's disease, Gilbert's syndrome, Reye's syndrome, Alagille syndrome, hemochromatosis, phenylketonuria and other aminoacidopathies, haemophilia and other clotting factor deficiencies, familial hypercholesterolemia and other lipid metabolism disorders, urea cycle disorders, fructosemia, glycogenosis, tyrosinemia, galactosemia, protein and carbohydrate metabolism deficiencies, organic aciduria, mitochondrial diseases, peroxysomal and lysosomal disorders, protein synthesis abnormalities, defects of liver cell transporters, defect of glycosylation, acute chemical toxicity, cholangitis, alpha-1-antitrypsin deficiency, biliary atresia, cystic disease of the liver, fatty liver, galactosemia, gallstones, porphyria, primary sclerosing cholangitis, sarcoidosis, tyrosinemia, or type 1 glycogen storage disease.

As used herein, the term “subject” refers to a mammalian animal, including, but not limited to, human, primates, domestic mammals, or laboratory mammals. A “subject” is preferably a human, but can also be an animal in need of treatment with an antagonist of BMP signaling, e.g., companion animals such as dogs, cats, and the like, farm animals such as cows, pigs, horses and the like and laboratory animals such rats, mice, guinea pigs and the like.

As used herein, the terms “treating” and “preventing” refers to preventing, blocking, suppressing, inhibiting, reducing, attenuating, ameliorating or reversing any or all conditions, effects or cause(s) of the liver injury, as well as symptoms or diseases related to the liver injury; increasing the time between the disappearance of a condition, symptoms or effect and its reoccurrence; stabilizing an adverse symptom associated with liver injury; or reducing, slowing, or stabilizing the progression of a condition associated liver injury.

As used herein, the term “transplantation” refers to grafting of one or more partial hepatic tissue(s) or cell(s) taken or derived from another or the subject's own liver.

As used herein, the term “resection” refers to the excision of a portion or all of an organ or other structure. For example, liver resection refers to surgical removal of a portion of the liver and is usually performed to remove the diseased portion of the liver, for example, liver tumors, that are located in the resected portion of the liver.

As used herein, the term “antagonist” refers to a chemical agent, pharmaceutical agent, a drug, a chemical compound, a small molecule, a protein, a peptide or an antibody that opposes the physiological effects of the BMP signaling pathway. For example, a chemical agent can opposes one or more BMP receptor-associated responses normally elicited by binding of one or more BMPs to one or more BMP receptors.

As used herein, the term “protein” refers to a polymeric form of any length of amino acids, which can include naturally-occurring or synthetic amino acids and coded and non-coded amino acids, peptides, depsipeptides, polypeptides with cyclic, bicyclic, depsicyclic, or depsibicyclic peptide backbones, single chain protein as well as multimers, as well as any fragment or portion of the intact protein molecule.

As used herein, “peptide” is meant any molecule composed of amino acids, amino acid analogs, chemically bound together. In general, the amino acids are chemically bound together via amide linkages (CONH); however, the amino acids may be bound together by other chemical bonds known in the art. For example, the amino acids may be bound by amine linkages. Peptide as used herein includes oligomers of amino acids, amino acid analogue, or small and large peptides, including polypeptides.

As used herein, the term “antibody” refers to an immunoglobulin or a fragment or a derivative thereof, and includes any polypeptide comprising an antigen-binding site, whether produced in vitro or in vivo. The term includes polyclonal, monoclonal, monospecific, polyspecific, humanized, single-chain, chimeric, synthetic, recombinant, hybrid, mutated, and grafted antibodies. The term “antibody” also includes antibody fragments such as Fab, F(ab′)₂, Fv, scFv, Fd, dAb, and other antibody fragments that retain antigen-binding function (i.e., the ability to bind a specific antigen). Typically, such fragments would comprise an antigen-binding domain (i.e., a part of an antibody molecule that comprises amino acids responsible for the specific binding between the antibody and the antigen). An antigen-binding domain typically comprises an antibody light chain variable region (V_{L}) and an antibody heavy chain variable region (V_{H}), however, it does not necessarily have to comprise both.

As used herein, the term “analogue” is a molecule whose structure is related to, but not identical to that of the corresponding antagonist. An analogue possesses a similar or analogous chemical and biological properties of the corresponding antagonist. For example, a chemical analogue retains the chemical activity of a corresponding antagonist, while having one or more chemical modifications. A polypeptide analogue retains the biological activity of a corresponding polypeptide antagonist, while having certain biochemical modifications from the polypeptide antagonist. Such modifications may increase, for example, the analogue's stability or half-life, without altering, for example, binding specificity to one or more biological molecules involved in the BMP signal transduction pathway. An analogue can include a synthetic chemical agent or polypeptide.

As used herein, an “effective amount” refers to an amount of the active antagonist of BMP signaling that is sufficient to affect the course and the severity of the liver injury, leading to the prevention, reduction, inhibition, attenuation or remission of liver injury. For example, an effective amount of an the BMP antagonist is an amount sufficient to inhibit BMP signaling in hepatocytes, leading to the prevention, reduction, inhibition, attenuation or remission of liver injury. As will be apparent to one skilled in the art, the effective amount for a given subject in need may vary according to the size, age, weight, condition and responsiveness of the subject to the treatment. The effective amount will also depend on the route of administration and the condition of the subject in need.

As used herein, the term “dose” refers to the quantity to be administered at one time, such as a specified and effective amount of the antagonist of BMP signaling.

As used herein, the term “modulate” or “regulate” refers to a change, such as an decrease or increase. For example, the change could refer to a biological activity. Desirably, the change is either an increase or a decrease of at least 10%, 20%, 30%, 40%, 50%, 60%, 70%, 80%, 90% or 95% in biological activity, relative to a reference or to control activity, for example, the biological activity of one or more SMADs, one or more BMPs, or one or more BMP receptors.

According to the present invention, administration of an antagonist of BMP signaling can enhance hepatocyte proliferation by inhibiting one of more BMP receptors. Inhibition of the BMP receptor(s) can be achieved by binding to one or more type I BMP receptors or one or more type II BMP receptors. Inhibition of the BMP receptor(s) can be achieved by simultaneously binding to one or more type I BMP receptors or one or more type II BMP receptors. The antagonist of the present invention can specifically inhibit a type I BMP receptor including, but not limited to, ALK2, ALK3 and ALK6. The type I BMP receptors include, but are not limited to, activin receptor-like kinase-2 (ALK2) (also known as activin receptor-I), ALK3 (also known as BMPR-IA), and ALK6 (also known as BMPR-IB). The type II BMP receptors include, but are not limited to, BMPRII, Act RIIa and ActRIIb. Both type I and type II BMP receptors transduce extracelluar BMP signals into the cytoplasm of the target cells. BMP ligands can facilitate assembly of the type I and II receptors to form a heterodimer and that the heterodimerization of the receptors allows the type II receptor to phosphorylate the type I receptor. When activated (“phosphorylated”), the type I BMP receptor phosphorylates BMP-responsive SMAD effectors (e.g., SMAD1, SMAD5 and SMAD8). When the type I BMP receptor is not phosphorylated, however, specific binding of a BMP to the type II BMP receptor elicits SMAD-independent signaling by activating the intracellular effectors including, but not limited to, mitogen-activated protein kinase (MAPK) p38.

The antagonist of the BMP pathway can inhibit BMP signaling by directly binding to one or more BMPs, particularly, BMP2, BMP3, BMP4, BMP5, BMP6 and BMP7. The antagonist of the BMP pathway can inhibit BMP signaling by binding directly to BMP2 or BMP4. The antagonist of the BMP signaling pathway can inhibit BMP signaling by inhibiting the interaction of BMP2 or 4 with a BMP receptor.

Because dimeric BMP ligands can facilitate assembly of the type I and II BMP receptors to form a heterodimer and that the heterodimerization of the receptors allows the type II receptor to phosphorylate the type I receptor, the antagonist of the BMP signaling can inhibit the interaction of a type II BMP receptor with a type I BMP receptor by hindering the binding interaction of one or more BMPs with the type I or type II receptor. The antagonist of the BMP signaling can also inhibit the interaction of a type II BMP receptor with a type I BMP receptor by impeding phosphorylation of the type I by the type II receptor.

The antagonist of BMP signaling can inhibit a BMP2 or BMP4-mediated signaling pathway by specifically binding to either BMP2 or BMP4 or by binding to a BMP2 or BMP4-specific BMP receptor. The antagonist of the BMP signaling pathway inhibits the activity of one or more BMPs. Preferably, the antagonist of BMP signaling inhibits the activity of one or more BMP by directly binding to one or more BMPs.

The invention includes methods of using an agent which inhibits one or more type I receptor. The antagonist of BMP signaling can inhibit BMP signaling by inhibiting phosphorylation of one or more SMADs by a type I BMP receptor. The type I receptor phosphorylates one or more receptor regulated SMADs (R-SMADs), including, but not limited to, SMAD1, SMAD2, SMAD3, SMAD4, SMAD5, SMAD8 or SMAD9. Preferably, the antagonist of the present invention inhibits phosphorylation of one or more SMADs including, but not limited to SMAD1, 5 or 8.

The antagonist of BMP signaling can be a pharmaceutical agent, a chemical agent, a protein, a peptide or an antibody. Preferably, the antagonist of the BMP signaling is a chemical agent that directly binds to one or more BMP receptors. Examples of suitable antagonists are described in WO 2008/033408, the entire teachings of which are incorporated herein by reference. Chemical agents used in the present invention include, but are not limited to, dorsomorphin, LDN193189 or an analogue or derivative thereof. Any BMP-specific inhibitor compound or chemical agent identified by the methods taught in WO 2008/033408 and evaluated for their ability to inhibit the BMP signaling pathway are contemplated in the present invention. The entire teachings of WO 2008/033408 are incorporated herein by reference.

Dorsomorphin is an inhibitor AMP kinase signaling pathway (Zhou et al., (2001) J. Clin. Invest. 108:1167-1174). Dorsomorphin is also shown to antagonize BMP signaling in zebrafish embryos and effectively promote dorsalization in zebrafish embryos (WO 2008/033408). In zebrafish, dorsomorphin is shown to inhibit the osteogenic differentiation induced by BMP4 or BMP6 in vitro, an inhibition more potent than that seen with treatment with noggin, a natural inhibitor of BMP signaling (WO 2008/033408). Dorsomorphin has been described to inhibit the activity of BMP2, BMP4, BMP6, BMP7 and BMP9 (WO 2008/033408) by selectively inhibiting type I BMP receptors, namely, ALK2, ALK3, and ALK6, and block BMP-mediated SMAD 1, 5 and 8 phosphorylation, without affecting MAPK p38 activation (Yu et al., (2007) Nature Chemical Biology, 4:33-41). Dorsomorphin contains the chemical structure as shown below in Structural Formula I:

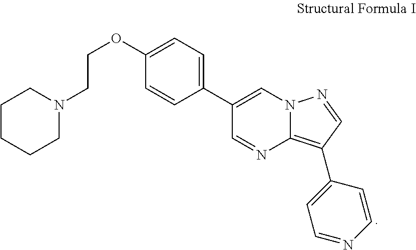

The biological function and structure of dorsomorphin, 6-[4-(2-piperidin-1-yl-ethoxy)-phenyl)]-3-pyrindin-4-yl-pyrazolo[1,5-a]-pyrimidine, are described in detail by Zhou et al. (Zhou et al. (2001) J. Clin. Invest. 108:1167-1174) and Yu et al. (Yu et al. (2007) Nature Chemical Biology, 4:33-41), the entire teachings of which are incorporated herein by reference.

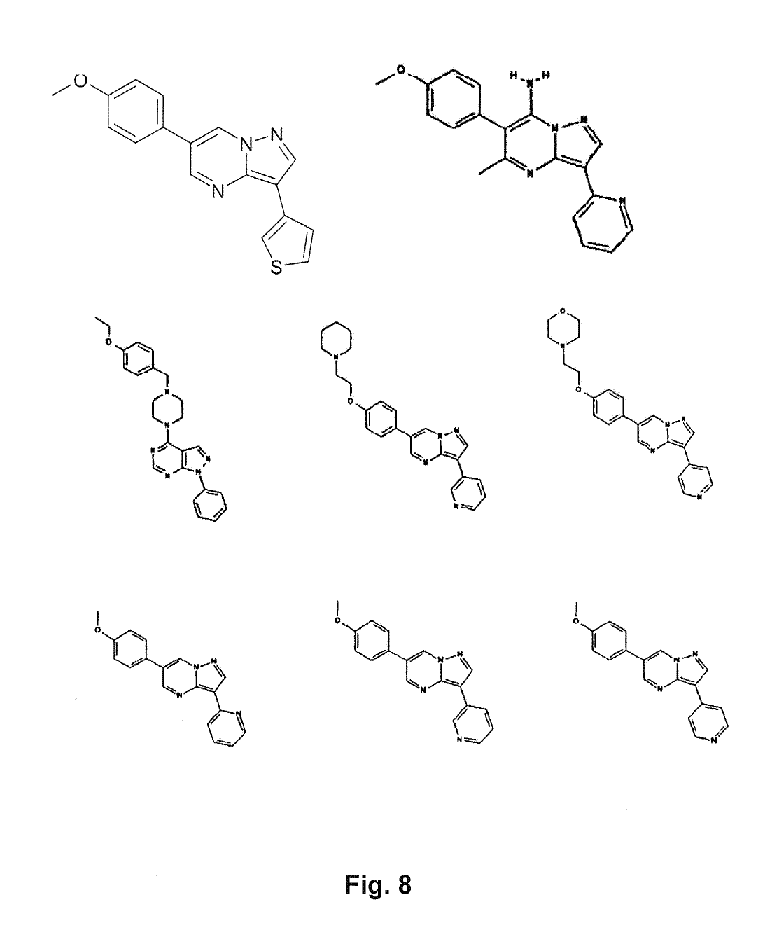

LDN193189 is a derivative of pyrazolo[1,5-a]-pyrimidine, which contains an amine on the 3- or 4-position of the pendent phenyl ring. Some pharmaceutically acceptable analogues of dorsomorphin are shown in FIG. 8 and also described in WO 2008/033408, the entire teachings of which are incorporated herein by reference. Methods of synthesizing LDN193189 and its analogues, including their molecular structures are described in detail by Cuny et al. (Cuny et al., Bioorganic & Medicinal Chemistry Letters, 18:4388-4392 (2008)), the entire teachings of which are incorporated herein by reference. It is also understood that one of ordinary skill in the art can readily prepare the analogues by using methods known in the art.

Biological impacts on liver regeneration of a chemical agent that modulates BMP signaling, for example, potential analogues of dorsomorphin or LDN193189 as well as other potential antagonist candidates, can be assessed by determining the effect of the agent on phosphorylation of SMAD 1, 5 or 8. BMP-mediated SMAD phosphorylation can be determined by, for example, immunoblot analysis using anti-phospho-SMAD 1, 5 or 8 (CELL SIGNALING®). Alternatively, BMP-mediated SMAD phosphorylation can be determined by immunoprecipitation and Enzyme Linked Immunosorbent Assay (ELISA) using permeabilized hepatocytes. Hepatocyte proteins are precipitated by, for example, methanol treatment using ice cold methanol for 10-15 minutes. Hepatocyte proteins are cross-linked with, for example, glutaraldehyde by introducing 0.25% to 2% glutaraldehyde and quenching it with an amine (e.g., lysine, ethylenediamine or phynylenediamine). Detailed protocols of phosphorylation assay is described in WO 2008/033408, the entire teachings of which are incorporated herein by reference. As used herein, detection of increased phosphorylation of SMAD indicates that the agent activates BMP signaling, whereas detection of decreased phosphorylation of SMAD indicates that the agent inhibits BMP signaling.

Alternatively, potential impacts on liver regeneration of a chemical agent that modulates BMP signaling can be determined by measuring hepatocyte proliferation using 5-Bromo-2-deoxy-uridine (BrdU) labeling or Ki-67 labeling as described herein.

The antagonists of BMP signaling can be present in pharmaceutically acceptable salt forms. Pharmaceutically acceptable salt forms include pharmaceutically acceptable acidic/anionic or basic/cationic salts. Pharmaceutically acceptable acidic/anionic salts include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphospate, polygalacturonate, salicylate, stearate, subacetate, succinate, sulfate, hydrogensulfate, tannate, tartrate, teoclate, tosylate, and triethiodide salts. Pharmaceutically acceptable basic/cationic salts include, the sodium, potassium, calcium, magnesium, diethanolamine, N-methyl-D-glucamine, L-lysine, L-arginine, ammonium, ethanolamine, piperazine and triethanolamine salts.

The BMP antagonist is administered to the subject in need anytime before, during or after liver injury. The BMP antagonist is administered before a liver injury. The BMP antagonist is administered anytime during the progression of liver injury. The BMP antagonist is administered after a liver injury. The BMP antagonist is administered in multiple doses at a number of intervals. The BMP antagonist can be administered at the time of 1, 2, 3, 4, 5, 6, 12, 24 and/or 48 hour(s) before the onset of a liver injury. The BMP antagonist can be administered at the time of and/or within 1, 2, 3, 4, 5, 6, 12, 24 and/or 48 hour(s) of the onset of the liver injury. The BMP antagonist can be administered at the time of and/or within 24, 36, 48, 60, 72, or 96 hours after a liver injury. The BMP antagonist can be administered at the time of and/or within 3, 4, 5, 6, 7, 8, 9, or 10 days after the onset of a liver injury. Alternatively, the BMP antagonist can be administered between 0-48 hours, 1-3 days, 2-4 days, 3-7 days, 5-8 or 7-10 days after liver injury. The BMP antagonist can be administered once during these time periods, or, alternatively, two, three, four, five, six, seven, eight, or even more times within these time periods. In one embodiment, treatment with a antagonist of BMP signaling ends after these time periods; alternatively, treatment can continue after the time period ends, for example, up to 1, 2, 3, 4, 5 or 6 months after the onset of the liver injury.

An antagonist of BMP signaling can be administered in an effective amount to a subject in need by any suitable route. For example, an effective amount of the antagonist of BMP signaling can be administered intravenously. An effectively amount of the antagonist of BMP signaling can be administered orally. The antagonist of BMP signaling can be administered by local introduction to the liver, for example, by hepatic catheterization. The antagonist of BMP signaling can be locally administered by infusion into the portal vein or other tributaries to the portal vein such as the lienal vein, the superior mesenteric vein, and the pyloric veins. Alternatively, the antagonist of BMP signaling can be locally administered to the hepatic tissue by injection. “Administered to the hepatic tissue by injection” means delivered to the inner or outer surfaces of the liver. In local administration or injection, the point of delivery of the BMP antagonist can be in sufficient proximity to the liver so that the BMP antagonist can diffuse and contact with at least one or more hepatocytes with the activated BMP signaling pathway in the liver. Preferably, the BMP antagonist can diffuse and contact with at least the majority (50% or more) of the hepatocytes capable of proliferating in the liver. The BMP antagonist can be administered to the subject in a sustained release formulation, or can be delivered by a pump or an implantable device, or by a carrier such as the polymers discussed below.

The antagonist of BMP signaling can be administered to the subject in conjunction with an acceptable pharmaceutical carrier as part of a pharmaceutical composition. The formulation of the pharmaceutical composition will vary according to the mode of administration selected. Suitable pharmaceutical carriers may contain inert ingredients which do not interact with the BMP antagonist. The carriers should be biocompatible, i.e., non-toxic, non-inflammatory, non-immunogenic and devoid of other undesired reactions at the administration site. Examples of pharmaceutically acceptable carriers include, for example, saline, commercially available inert gels, or liquids supplemented with albumin, methyl cellulose or a collagen matrix. Further examples include sterile water, physiological saline, bacteriostatic saline (saline containing about 0.9% mg/ml benzyl alcohol), phosphate-buffered saline, Hank's solution, Ringer's-lactate and the like. Standard pharmaceutical formulation techniques can be employed, such as those described in Remington's Pharmaceutical Sciences (Remington's Pharmaceutical Sciences. XVIII, Mack Publishing Company, Easton, Pa. (1990)).

The antagonists of BMP signaling can be administered in a sustained release formulation. Polymers are often used to form sustained release formulations. Examples of these polymers include poly α-hydroxy esters such as polylactic acid/polyglycolic acid homopolymers and copolymers, polyphosphazenes (PPHOS), polyanhydrides and poly (propylene fumarates). Polylactic acid/polyglycolic acid (PLGA) homo and copolymers are well known in the art as sustained release vehicles. The rate of release can be adjusted by the skilled artisan by variation of polylactic acid to polyglycolic acid ratio and the molecular weight of the polymer (see Anderson et al. (1997) Adv. Drug Deliv. Rev. 28:5, the entire teachings of which are incorporated herein by reference). The incorporation of poly-ethylene glycol into the polymer as a blend to form microparticle carriers allows further alteration of the release profile of the active ingredient (see Cleek et al., J. Control Release 48:259 (1997), the entire teachings of which are incorporated herein by reference). Ceramics such as calcium phosphate and hydroxyapatite can also be incorporated into the formulation to improve mechanical qualities.

The invention is illustrated by the following example which are not intended to be limiting in any way.

### Example 1

### I. Experimental Procedures

The present invention is based on a discovery that the bone morphogenetic protein (BMP) signaling pathway is an active inhibitor of hepatocyte proliferation and that treatment with an antagonist of the pathway results in enhancement of hepatocyte proliferation and increased liver regeneration after liver injury in vivo.

A. Experimental Procedures for Partial Hepatectomy

Animals were used to examine the role of various members of BMP signaling in restoration of liver mass after injury. Mice were subjected to two-thirds (“⅔”) or massive eighty percent (80%) hepatectomy. Surgery was performed on adult mice at least 8 weeks of age. For surgery, mice were anesthetized with 100 mg/kg ketamine and 10 mg/kg xylazine which were injected intraperitoneally. In the event that the subject did not become unconscious, an extra 25 mg/kg ketamine and 2.5 mg/kg xylazine was injected in a similar fashion. This was repeated until the subject was unconscious. Hair was removed from the surgical site using a clipper and the skin was disinfected using aseptic technique and betadyne. The scrubbed area was rinsed with 70% alcohol. To avoid hypothermia, the animal was not wetted more than necessary. An incision was made with a sterile scissors at the level of the xiphiod approximately one centimeter toward the umbilicus. The peritoneum was entered with a scissors and the tip of the xiphoid was excised. For ⅔ hepatectomy, the liver was exteriorized through the incision via gentle pressure on the flanks over an area previously prepped. A 4-0 silk ligature was placed around the exteriorized portion of the liver and tied. The anterior portion of the liver distal to the tie was then excised. A 4-0 proline was used to close the fascia and a 4-0 nylon was used to close the skin in a subcuticular fashion. For 80% hepatectomy, a larger incision was used and the individual lobes tied with a 4-0 silk ligature, leaving only the caudate. The anterior portions of the liver distal to the tie was then excised. A 4-0 proline was used to close the fascia and a 4-0 nylon was used to close the skin in a subcuticular fashion.

B. Genotyping and Selection of Subjects

Tail biopsies were performed on mice 2 weeks of age for genotyping by removing approximately 2 mm of the tail. Sterile equipment was used for all procedures. The distal portion of the tail was wiped with 70% alcohol and snipped using scissors. Firm pressure was applied to the cut end for at least 30 seconds. Verification of bleeding cessation was noted before returning the mice to the cage. In case where bleeding occurred, one drop of NEXABAND® Liquid was applied to cut end of tail. Male adult mice was used for the experiments, with 5 animals for each time point. Inbred mouse strains was used to decrease experimental variability.

C. Determination of Expression Levels of BMP mRNA and Proteins in the Liver

Expressions of BMP mRNA and proteins were significantly decreased in the liver following partial hepatectomy. First, endogenous gene expression was determined by measuring the level of mRNA expression (e.g., expression level of target BMP4 gene). The level of gene expression was measured using, for example, polymerase chain reaction (PCR), ligase chain reaction (LCR), or hybridization assays such as Northern hybridization, dot blotting and RNase protection. Particularly, quantitative real time polymerase chain reaction (RT-PCR) was utilized to quantitate the level of BMP4 mRNA after hepatectomy. The level of protein or mRNA was detected using directly or indirectly labeled detection agents, e.g., fluorescently or radioactively labeled nucleic acids, radioactively or enzymatically labeled antibodies, and the like. Methods for detecting the level of protein and mRNA are described in U.S. Pat. No. 5,210,015 to Gelfand, U.S. Pat. No. 5,538,848 to Livak, et al., and U.S. Pat. No. 5,863,736 to Haaland, as well as Heid, C. A., et al., Genome Research, 6:986-994 (1996); Gibson, U. E. M, et al., Genome Research 6:995-1001 (1996); Holland, P. M., et al., Proc. Natl. Acad. Sci. USA 88:7276-7280, (1991); and Livak, K. J., et al., PCR Methods and Applications 357-362 (1995). The entire teachings of these reference are incorporated herein by reference.

D. Creation of a Transgenic Mouse Exhibiting Conditional BMP4 Deletion in the Liver

Complete deletion of BMP4 has been known to be embryonic lethal. Accordingly, a strategy of a liver-specific conditional gene deletion (“knock-out) for a BMP was employed. Generally, in the knock-out system, alteration of the target gene occurs upon exposure of the animal to a substance that promotes target gene alteration, for example, introduction of an enzyme that promotes recombination at the target gene site (e.g., Cre in the cre-flox system). In the present invention, a floxed BMP4 mouse was provided and cre-recombinase was delivered either via an AAV-8-MUP-cre (Ho et al., 2008) or a transgenic mouse expressing Cre-recombinase under the control of the albumin and alpha-fetoprotein promoters (Alfp-Cre). As a research technology, use of viral vectors to study the liver has great utility. Unfortunately, lentiviruses and adenoviruses have limited utility due to difficulty in administration, inflammation, or liver toxicity (Mahasreshti et al., 2003). To overcome this problem, an adeno-associated virus 8 with a mouse urinary protein promoter (AAV-8-MUP), which infects the liver with little damage and no effect on repair after injury, was developed and employed.

E. Antibody Labeling of Ki-67

To examine the functional consequences of loss of BMP4 signaling, Ki-67 labeling indices were employed to determine hepatocyte proliferation in BMP null mice versus wild-type. Adult male mice 8-10 weeks of age underwent ⅔ hepatectomy as described above and hepatocyte proliferation was examined at various time points (e.g., 0, 24, 48, 72, 96, 120, 144, 168 and 192 hours) following ⅔ hepatectomy. As a cellular marker for proliferation, Ki-67 protein is strongly associated with cell proliferation and division. During interphase, the Ki-67 protein (antigen) is exclusively detected within the nucleus, whereas, during mitosis, most of the protein localize to the chromosomes. Ki-67 protein is detected during all active phases of the cell cycle (G₁, S, G₂, and mitosis), but is completely absent from resting cells (G₀). Thus, Ki-67 serves as an excellent marker for determining the growth fraction of a given cell population. The fraction of Ki-67-positive cells (the Ki-67 labeling index) is correlated with the course of cell division and growth. The monoclonal antibody, MIB-1, was employed to detect the Ki-67 antigen in the hepatocytes extracted from the mice. Ki-67 antibody labeling index including immunostaining was performed as described by Suzuki et al. (Suzuki et al. (1992) J. Clin. Gastroenterol. 15:317-20), the entire teachings of which are incorporated herein by reference. A variety of controls were run, including Alfp-Cre/BMP4^{f/f} and Alfp-Cre⁺BMP4^{+/f} mice, and mice receiving AAV-8-MUP-Green fluorescent protein (GFP) control virus.

F. 5-Bromo-2-deoxy-uridine (BrdU) Labeling and Immunocytochemistry

The liver and hepatocytes were extracted from the mice as described above and grown in T flasks in traditional DMEM with 10% FBS. Cells were dissociated using trypsin/EDTA solution, and transfer to shell vials with 12 mm coverslips in 1 mL of appropriate media or, alternatively, polylysine-coated coverslips (0.05-0.1% in PBS, overnight using 150-300K mol. wt) in 24 well plates or chamber-slides. Cell were incubate cells at 37° C. for 2 hours to allow for recovery. BrdU was added to each shell vial to a final concentration of 10 μM and incubate at 37° C. for 2 hours. Medium was aspirated out from the shell vials and the coverslips were washed 3× in PBS. 1 mL of Carnoy's fixative (3 parts methanol:1 part glacial acetic acid) was added to each shell vial and aspirated out. An additional 1 mL of Carnoy's fixative was immediately added. Shell vials were fixed at −20° C. for 20 minutes. The fixative was aspirated from the shell vials and coverslips were washed 3× in PBS. Cells were denatured by adding 0.2 mL of 2M HCl in water to each shell vial and by incubating at 37° C. for 1 hour. Acid was aspirated out from the shell vials and coverslips were neutralized by washing 3× in borate buffer pH 8.5. The shell vials were washed 3× in PBS+0.05% Tween 20 [PBS/T20] and stored at 2-8° C.

Immunostaining

Immunostaining was performed as generally known in the art. Specifically, cells were blocked by adding 0.2 mL of PBS/T20/2% normal goat serum to each vial and incubating at 37° C. for 10-30 minutes. Solution was aspirated out and 0.2 mL of anti-BrdU monoclonal antibody diluted in PBS/T20/2% normal goat serum (Millipore™ catalog no. MAB3424, MAB4072) was added. Vials were incubate at 37° C. for 30 minutes (BrdU stock solution: 10 mM dissolved in PBS, 0.2 μm filter sterilized and stored at −20° C.). BrdU stock solution was diluted 1/100 in culture medium to yield a 100 μM solution (10×). The 10× solution was added to each vial to a final concentration of 10 μM. Vials were washed 3× in PBS/T20, and 0.2 mL of goat anti-mouse FITC (Millipore™ catalog no. AP124F) was added. Vials were incubated at 37° C. for 30 minutes in the dark and washed 3× in PBS/T20. Coverslips were removed from the vials, dipped in DI water and mounted on glass slides, using mounting media for fluorescence (Millipore™ catalog no. 5013). Slides were evaluated using fluorescence microscopy.

### II. Results

A. BMP4 is Constitutively Expressed by the Adult Liver, and Serves as a Check on Liver Regeneration and Removing this Inhibition Using a Variety of Methods Enhances Liver Regeneration

A large-scale genetic screen identified bone morphogenetic protein signaling as highly inhibited during liver repair after injury. This included induction of antagonists and inhibition of agonists. These molecules as potential targets to understand a new paradigm in liver regeneration in which the antagonism of inhibitors of proliferation is required for normal liver repair after injury. Since BMPs are secreted signaling molecules and therefore potentially able to manipulated, this was a particularly attractive therapeutic target.

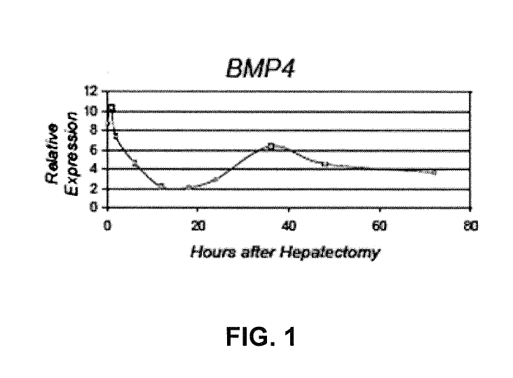

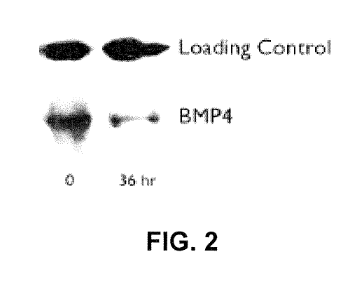

Real time PCR for BMP4 after hepatectomy is shown in FIG. 1. By 18 hours after hepatectomy, the level drops to 25% compared with the uninjured liver. BMP4 protein levels decrease in a similar fashion, and are down significantly 36 hours after hepatectomy (FIG. 2). Similar results were obtained for BMP2 RNA.

B. Expression of BMP4 Receptors in the Liver

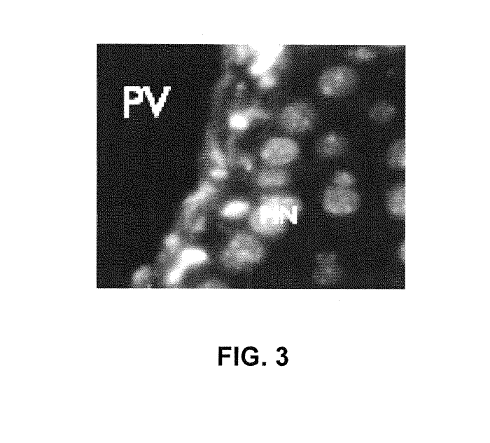

To determine that receptors for BMPs are present in the liver, immunohistochemistry was performed for ALK2 and ALK3, two receptors utilized by BMP4. ALK2 is mainly utilized by BMP6 and BMP7, whereas ALK3 is mainly utilized by BMP2 and BMP4 as discussed above. As shown in FIG. 3, ALK3 is expressed in the liver adjacent to the portal vein (red staining) Similar results were obtained for ALK2.

C. Creation of Conditional Gene Deletion of BMP4 in Mouse Liver

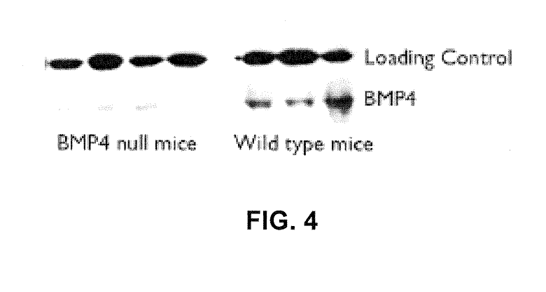

Complete deletion of BMP4 has been known to be embryonic lethal. Accordingly, a strategy of a liver-specific conditional gene deletion (“knock-out) for a BMP was employed. Specifically, a floxed BMP4 mouse with Cre-recombinase delivered either via an AAV-8-MUP (Ho et al., 2008) or a transgenic mouse expressing Cre-recombinase under the control of the albumin and alphafetoprotein promoters (Alfp-Cre) was employed. To confirm the effectiveness conditional deletion of BMP4 in the liver, western blot assay for BMP4 protein was performed after recombination. As depicted in FIG. 4, drastically reduced levels of BMP4 proteins were observed in floxed mice acted on by Cre-recombinase (FIG. 4).

D. Enhancement of Liver Regeneration by Loss of BMP4

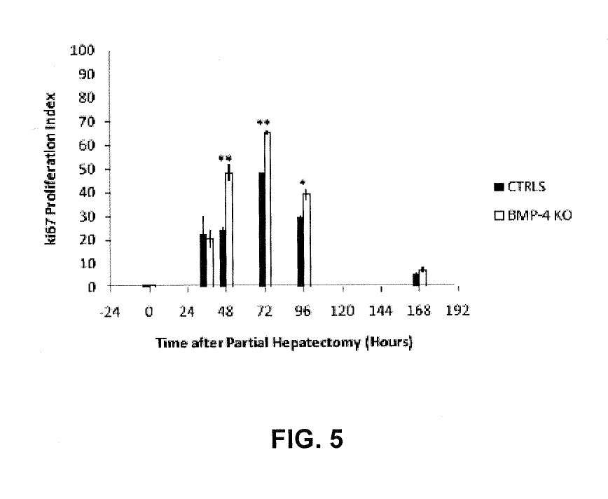

Next, the functional consequences of loss of BMP4 signaling were examined by using Ki-67 labeling indices. Adult male mice 8-10 weeks of age underwent ⅔ hepatectomy as described above and hepatocyte proliferation was examined at various time points (e.g., 0, 24, 48, 72, 96, 120, 144, 168 and 192 hours) following ⅔ hepatectomy. As shown in FIG. 5, liver-specific deletion of BMP4 in the transgenic mice results in more than double the amount of hepatocyte proliferation at 48 hours as compared to controls as measured by Ki-67 labeling (FIG. 5) (p<0.05). A variety of controls were run with similar results, including Alfp-Cre/BMP4^{f/f} and Alfp-Cre⁺BMP4^{+/f} mice, and mice receiving AAV-8-MUP-Green fluorescent protein (GFP) control virus (not all data shown). Therefore, loss of BMP4 in the liver significantly enhanced liver regeneration by releasing a constitutive block for liver regeneration.

E. Reduction of BMP4 Expression for Normal Liver Repair After Liver Injury

To demonstrate that reduction or inhibition of BMP4 signaling is a requirement for normal liver repair after liver injury, it was necessary to maintain BMP4 expression levels after hepatectomy. To accomplish this, an AAV-8-MUP-BMP4 was developed. A 70% decrease in hepatocyte proliferation was observed when BMP4 virus is expressed during and after hepatectomy (P<0.05) compared with vehicle (saline) or AAV-8-MUP-GFP control virus.

To establish that liver-specific loss of BMP4 did not produce systemic effects that would influence liver regeneration, the BMP4 virus (i.e, AAV-8-MUP-BMP4) was injected into liver-specific BMP4 null mice. As expected, expression of BMP4 in the liver returned hepatocyte proliferation to the lower, normal levels seen in the wild type mice.

These results strongly demonstrate that reduction of BMP signaling is necessary for normal hepatocyte proliferation after hepatectomy. Furthermore, these results provide strong evidence that the extent of BMP4 signaling controls the rate of hepatocyte proliferation after hepatectomy. These results are also consistent with the observation that release of inhibitory pathways is a critical mechanism for liver renewal after injury as shown in FIG. 5. Since BMP4 is a secreted signaling molecule, it serves as an excellent candidate for a therapeutic target.

F. Enhancement of Liver Regeneration by Pharmacological Inhibition of BMP Signaling after Partial Hepatectomy

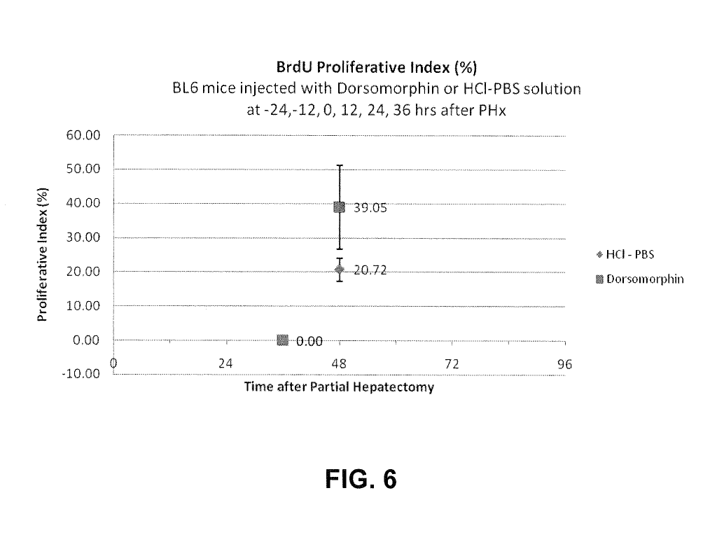

To provide clinical relevance for the findings discussed above, eight week old mice were injected with an antagonist of BMP signaling. Dorsomorphin was injected for 48 hours prior to, during, and after ⅔ hepatectomy. Specifically, dorsomorphin was injected multiple times at 24 and 12 hours prior to hepatectomy, at the time of hepatectomy, and 12, 24, and 36 hours after hepatectomy. In FIG. 6, BrdU incorporation was measured at 36 and 48 hours after ⅔ hepatectomy. As demonstrated in FIG. 6, administration of dorsomorphin resulted in marked enhancement of hepatocyte proliferation. Dorsomorphin administration for up to 60 days did not produced systemic toxicity. In particular, there was no effect on bone marrow, growth, or white blood cell count.

### Example 2

### I. General Approach to Assessing Liver Regeneration after Hepatectomy and Tylenol Overdose

Three experimental techniques will be employed to examine effects of the genetic or pharmacological interventions: ⅔ hepatectomy, massive hepatectomy, or Tylenol overdose. For ⅔ hepatectomy, removal of the left and middle lobes including the gallbladder will be performed as described above and described by others (Otu et al., 2007). For massive hepatectomy (80%), the right lobe will also be resected. For Tylenol overdose, 300 mg/kg will be administered by intravenous perfusion.

To analyze the ⅔ hepatectomy animals, livers will be harvested at 6, 12, 24, 36, 48, and 72 hours, and 7 days after hepatectomy. BrdU will be injected 2 hours prior to animal sacrifice and BrdU incorporation as well as Ki-67 labeling will be used to determine hepatocyte proliferation. Liver weights will be analyzed at the time of sacrifice and liver/body weight ratio will be determined to assess the speed of regeneration. RNA and protein samples will be collected for real-time PCR analysis and Western blotting. In general, whether the initiation of proliferation after hepatectomy is delayed or accelerated will be assessed. It will be also determined whether the pace of regeneration is altered after it begins, and whether the set point for the final liver size is altered. Histology will be also examined to look for fat accumulation, vacuolization, necrosis, or architectural changes in the liver. To further assess hepatocyte health and function, liver function tests including ast, alt, and bilirubin will be measured at baseline and in the sacrificed animals. To assess potential levels of programmed cell death (apoptosis) or necrosis in the response to liver injury, terminal deoxynucleotidyl transferase dUTP nick end labeling (TUNEL) will be performed to analyze DNA fragmentation. As discussed above, male mice of 8-10 weeks old will be employed in all experiments, using littermates as controls wherever possible. For survival experiments, Kaplan-Meier survival curves will be generated. 22 experimental mice will be compared with 22 control mice based on power calculations.

### Example 3

### I. In Vivo Studies to Determine which BMP Ligands and Receptors Affect Liver Repair after Injury and Elucidate their Molecular Mechanism of Action

A. Determining the Mechanism of Enhanced Liver Repair after Injury in Liver-Specific BMP Null Mice

Controls with wild type mice, wild type mice with the virus, transgenic floxed mice receiving a control AAV-8-MUP-EGFP virus as well as experimental transgenic floxed mice receiving a AAV-8-MUP-BMP virus are described above. The temporal signature of the enhanced hepatocyte proliferation after hepatectomy in liver-specific BMP4 null mice is depicted in FIG. 5.

To further determine more specific mechanism of enhanced liver repair after injury in liver-specific BMP4 null mice and to evaluate the mechanism by which BMP4 is repressing proliferation, the following studies will be performed.

It is likely that there is a feedback loop for BMP expression as a number of antagonists exist. To determine whether there exists a feedback loop for BMP signaling, it will be determined how other components of the BMP pathway, for example, BMP2, BMP6 or BMP7, type I BMP receptors such as ALK2, ALK3, ALK6 and antagonists, such as Noggin and Cerberus-like 1, change over time in the absence of BMP4 by using RT-PCR and Western blotting.

BMP4 is likely to increase cyclin expression and as an upstream effector of these proteins. To assess how loss of BMP4 affects cell cycle progression, cyclin A and D will be investigated by, for example, RT-PCR and Western blotting.

Although it is likely that c-jun and c-EBP-α are not transcriptional targets of BMP4, it will be important to confirm the relationship. To examine how loss of BMP4 affects the transcriptional regulation of positive regulatory pathways, expression of c-jun and c-EBP-α will be determined by, for example, RT-PCR and Western blotting.

It is likely that cytokines and growth factors are not targets of BMP4. To determine whether BMP4 regulates other stimulators of hepatocyte proliferation, expression of the cytokine IL-6 and growth factors epidermal growth factor and hepatocyte growth factor will be examined by, for example, RT-PCR and Western blotting. It will be also examined whether BMP4 signaling transcriptionally regulates the counter-regulatory proteins p21 and p53 by RT-PCR or northern analysis.

Localization and their phosphorylation state of the intracellular mediators of BMP signaling including SMAD 1, 5 and 8 will be also determined by using Western blotting.

Tob1 may function as a transcriptional target of BMP4. Changes in transducer of ErbB2.1 (Tob1), an anti-proliferative molecule that interacts with BMP signaling, will determine if BMP signaling modulates Tob1 expression.

B. Determining Whether Liver-Specific Removal of BMP2 Enhances Liver Repair after Injury

Since traditional knockout of BMP2 is embryonic lethal, a strategy using ‘floxed’ BMP2 (BMP2^{f/f}) mice will be employed similar to the strategy used for BMP4 described above. Inactivation of BMP2 in the BMP2^{f/f} can be performed in two separate ways, using a viral or transgenic approach. The AAV-8-MUP-Cre is preferable since the recombination occurs only on injection of the mice, and not prior to birth. The temporal specificity that can be achieved with the virus means less exposure of the animal to low BMP2 levels which might complicate the analysis. Controls for the virus-based approach will be similar to those for BMP4 and based on BMP2^{f/f} and an eGFP virus to eliminate the possibility that the floxed but non-recombined allele, or the virus itself is affecting the mice.

Verification of successful gene deletion will be performed in separate PCR assays to look for the recombination event. These assays are routinely performed by one having ordinary skill in the art. Western blot assays will be performed as an additional control to ensure deletion of BMP2 similar to the assay shown in FIG. 4.

To determine whether the conditional null mice have a phenotype in the absence of hepatectomy. Liver weights will determine if BMP2 regulates liver size. Liver function tests and blood counts will be determined for any possible effects on the liver and bone marrow. Since SMADs mediate BMP signaling, and SMAD4 has been shown to have a role in iron metabolism via hepcidin expression (Wang et al., 2005), additional studies of these mice will be performed for the levels of iron, transferrin and ferritin as described in Yu et al. (Yu et al. (2007) Nature Chemical Biology, 4:33-41), the entire teachings of which are incorporated herein by reference.

Liver-specific BMP2 null mice will be generated by infection of floxed BMP2 mice with AAV-8-Cre with controls as described above. These mice will be examined for hepatocyte proliferation between 6 hours and 7 days following ⅔ hepatectomy. To determine the mechanism of action, similar analysis to the BMP4 null mice will be performed for BMP2 null mice using real-time PCR and Western blotting. To further determine whether deletion of BMP2 leads to enhanced hepatocyte proliferation after hepatectomy, BrdU incorporation and Ki-67 staining will be also performed using BMP2 null mice. Any differences in proliferation between BMP2 and BMP4 mice will point to specific functions of BMP2 versus BMP4, suggesting how different members of the pathway are responsible for different functions.

C. Determining Whether Liver-Specific Removal of ALK2/ALK3 Enhances Liver Repair after Injury

Whether removal of Alk3 increases hepatocyte proliferation after hepatectomy and lead to more rapid restoration of liver mass will be determined in liver-specific Alk3 null mice. The goal is to characterize the effect of loss of ALK3, a BMP receptor, on liver repair after injury. In identifying a pharmaceutical antagonist of high specificity, it is critical to determine which of the BMP receptors are mediating the relevant signal. A “floxed” ALK3 (AIk3^{f/f}) mice will be created as described above. To determine whether the conditional null mice have a phenotype in the absence of hepatectomy, a broad survey of these mice will be performed to determine the levels of iron, transferrin, ferritin, hematocrit, and evidence of anemia or abnormal red cell morphology.

Similar to BMP4, inactivation of ALK3 can be performed in two separate ways: the virus approach and transgenic approach. Controls for the virus-based approach will use ALK3^{f/f} with an eGFP virus to eliminate the possibility that the floxed but non-recombined allele has systemic effects that could complicate the analysis, or the virus itself is affecting regeneration.

Gene deletion will be verified using PCR assays to look for the recombination event and Western blot to ensure protein removal. Liver-specific Alk3 null mice generated by infection with AAV-8-MUP-Cre will be compared to floxed Alk3 mice receiving AAV-8-MUP-GFP control virus. These mice will be examined for hepatocyte proliferation after ⅔ hepatectomy between 6 hours and 7 days after hepatectomy. To determine the mechanism of action, similar analysis to the BMP4 null mice will be performed using real-time PCR and Western blotting. If the liver mass is still different from controls after 7 days, it will suggest that Alk3 may be involved in longer-term modulation of liver size.

Similarly, liver-specific Alk2 single knock-out as well as Alk2/AIk3 double knock-out (“null”) mice will be employed using the similar method employed to determine the specific molecular mechanisms of the these components in liver regeneration.

### Example 4

### I. Determination of Whether a Pharmaceutical or Chemical Agent that Inhibits BMP Signaling can Improved Hepatocyte Proliferation after Partial Hepatectomy, Enhance Liver Repair after Injury, and Improve Survival after Massive Hepatectomy or Acetaminophen (Tylenol®) Overdose

A. Determining Whether a Pharmaceutical or Chemical Agent that Inhibits BMP Signaling can Enhance Liver Repair after Injury (⅔ Hepatectomy)

C57B6 mice will undergo a series of dosing regimens using dorsomorphin or LDN193189, selective inhibitors of BMP signaling. Other compounds that are described in WO 2008/033408 or compounds that are identified by the methods describe in WO 2008/033408 will be also employed. First, a dose response curves for the liver for these compounds will be produced. Since inhibition of BMP signaling is known to influence Id-1 and hepcidin expression, real time PCR and Western blotting will be performed as an independent output to assess the efficiency of the pharmaceutical block. To determine whether there are systemic effects of the compounds, liver function tests, iron, ferritin levels will be also examined. The appropriate doses will be determined as the lowest dose that produces maximal enhancement of hepatocyte proliferation after ⅔ hepatectomy (“partial hepatectomy”). Once this is determined, mice will undergo ⅔ hepatectomy after dosing with dorsomorphin or LDN193189. Livers will be harvested at 6, 24, 36, 48, 72, 96, and 168 hours. Control mice will be treated with saline alone. The mechanism by which dorsomorphin influences hepatocyte proliferation will be assessed by BrdU incorporation and Ki-67 labeling assays as described above. Similarly, real-time PCR and Western blotting will be also performed to determine the levels of BMP expression as discussed above.

B. Determining Whether Dorsomorphin or LDN193189 Enhances Survival after Massive Liver Resection (80% Hepatectomy)

Using a variety of dosing regimens as described, C57/B6 mice will undergo 80% hepatectomy as described above. Mice administered with dorsomorphin or LDN193189 will be compared with mice receiving saline as a control. Kaplan-Meier curves will be generated. Livers will be harvested in survivors at 48 and 96 hours, and then at 7 days for histology to evaluate hepatocyte health and BrdU incorporation to assess hepatocyte proliferation. TUNEL labeling will be used to evaluate the levels of apoptosis. A 30% survival without intervention is expected based on the prior observation. Power calculations suggest 22 control mice and 22 receiving drug will be adequate.

C. Determining Whether Dorsomorphin or LDN193189 Enhances Survival after Acetaminophen (Tylenol®) Overdose

Mice will be administered acetaminophen (Tylenol®) at 300 mg/kg after receiving dorsomorphin or LDN193189. The dose will be adjusted to produce 70% mortality in the experimental animals. Control animals receiving saline will serve as controls. At least 22 control mice and 22 experimental mice will be employed. Dose-response curves for survival will be generated. Histology will be performed at 48 and 96 hours, and again at 7 days to evaluate hepatocyte health and hepatocyte proliferation will be assessed by BrdU assay. Apoptosis will be evaluated using TUNEL staining.

Although it is likely that enhancing hepatocyte proliferation can tip the balance to survival, hepatocyte necrosis from acetaminophen (Tylenol®) may make the hepatocytes unable to respond to the proliferative stimulus. In humans, hepatocytes proliferate after acetaminophen (Tylenol®) toxicity, but, in the absence of life support for the mice, this may not be feasible.

### Example 5

### Mouse Study

LDN (3 mg/kg) was injected intraperitoneally (IP) twice per day for 2 days prior to 70% hepatectomy and then twice/day for 2 days after hepatectomy. Two hours prior to sacrifice, bromo-deoxyuridine (100 mg/kg) was administered IP. Animals were sacrified at 48 hours after hepatectomy. Liver sections were cut and subjected to immunohistochemical analysis for BrdU. Stained hepatocytes were counted and a ratio of stained to unstained hepatocytes provided the percentage of cells proliferating.

Results: Hepatocyte proliferation 48 hours after hepatectomy with LDN versus control: 77.2+/−11.1 vs. 34.5+/−10.2 cells/high power field.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. An antagonist of a bone morphogenetic protein (BMP) signaling pathway, for use in liver treatment.

2. An antagonist according to claim 1, for use in treating liver injury or disease.

3. An antagonist according to claim 1, for the use described therein; wherein said treatment is preventative treatment.

4. An antagonist according to claim 1, for the use described therein; wherein the antagonist increases proliferation of hepatocytes.

5. An antagonist according to claim 1, for the use described therein; wherein the antagonist inhibits or down regulates a constitutively active BMP signaling pathway.

6. An antagonist according to claim 5, for the use described therein; wherein said BMP signal pathway is a BMP2 or BMP4-mediated signaling pathway.

7. An antagonist according to claim 5, for the use described therein; wherein said antagonist of said BMP signaling pathway inhibits type I BMP receptor signal transduction.

8. An antagonist according to claim 7, for the use described therein; wherein said antagonist of said BMP signaling pathway inhibits type I BMP receptor signal transduction by binding to said type I receptor.

9. An antagonist according to claim 7, for the use described therein; wherein said type I BMP receptor is ALK2, ALK3 or ALK6.

10. An antagonist according to claim 9, for the use described therein; wherein said type I BMP receptor is ALK2 or ALK3.

11. An antagonist according to claim 5, for the use described therein; wherein said antagonist of said BMP signaling pathway inhibits phosphorylation of smad-1, 5 or 8.

12. An antagonist according to claim 5, for the use described therein; wherein said antagonist of said BMP signaling pathway inhibits binding of BMP2 or 4 to a BMP receptor or interaction of a type II BMP receptor with a type I BMP receptor.

13. An antagonist according to claim 5, for the use described therein; wherein the antagonist is a chemical agent.

14. An antagonist according to claim 13, for the use described therein; wherein the chemical agent is dorsomorphin, LDN193189, or an analogue of any of the foregoing.

15. An antagonist according to claim 1, for use in liver regeneration.

16. An antagonist according to claim 1, for use in treating partial loss or injury to the liver.

17. An antagonist according to claim 16, for the use described therein; wherein said loss or injury is caused by a surgical or transplantation procedure.

18. An antagonist according to claim 17, for the use described therein; wherein said surgical procedure is liver resection.

19. An antagonist according to claim 17, for the use described therein; wherein the loss or injury is liver insufficiency

20. An antagonist according to claim 16, for the use described therein; wherein said treatment precedes said injury.

21. An antagonist according to claim 1, for the use described therein; wherein said use is in respect of treatment of a mammal.

22. An antagonist according to claim 1, for the use described therein; wherein said use is in respect of treatment of a human.

23. An antagonist according to claim 21, for use described therein; wherein said use is in respect of the treatment of a liver transplantation donor.

24. An antagonist according to claim 21, for the use described therein; wherein said use is in respect of the treatment of a liver transplantation recipient.

25. An antagonist according to claim 2, or any claim dependent thereon, for the use described therein; wherein said liver injury is caused by a hepatotoxic chemical agent.

26. An antagonist according to claim 25, for the use described therein; wherein said hepatotoxic chemical agent is acetaminophen.

27. An antagonist according to claim 1, for use in treating a liver injury caused by a liver disease selected from the group consisting of hepatitis A, hepatitis B, hepatitis C, other hepatitis viral infections, autoimmune hepatitis, cirrhosis, biliary cirrhosis, acute liver failure, chronic liver failure, acute liver failure, acute liver infection, cancer, Wilson's disease, Gilbert's syndrome, Reye's syndrome, Alagille syndrome, hemochromatosis, phenylketonuria and other aminoacidopathies, haemophilia and other clotting factor deficiencies, familial hypercholesterolemia and other lipid metabolism disorders, urea cycle disorders, fructosemia, glycogenosis, tyrosinemia, galactosemia, protein and carbohydrate metabolism deficiencies, organic aciduria, mitochondrial diseases, peroxysomal and lysosomal disorders, protein synthesis abnormalities, defects of liver cell transporters, defect of glycosylation, acute chemical toxicity, cholangitis, alpha-1-antitrypsin deficiency, biliary atresia, cystic disease of the liver, fatty liver, galactosemia, gallstones, porphyria, primary sclerosing cholangitis, sarcoidosis, tyrosinemia, and type 1 glycogen storage disease.

28. A method performed outside the human or animal body comprising administering an antagonist to a liver, to a part of a liver, or to hepatocytes; wherein said antagonist is an antagonist according to claim 1.

29. A composition comprising a liver, a part of a liver, or hepatocytes, said composition further comprising an antagonist; wherein said antagonist is an antagonist as described in any preceding claim and wherein said liver, part of a liver, or hepatocytes is/are not located within the human or animal body.

30. A composition according to claim 29; for use in transplantation.
